(19) United States
(12) Patent Application Publication
(10) Pub. No.: US 2026/0100245 A1
(43) Pub. Date: Apr. 9, 2026

(54) METHOD, ELECTRONIC DEVICE AND STORAGE MEDIUM

(71) Applicants: Bytedance Technology Ltd., Grand Cayman (KY); Beijing Zitiao Network Technology Co., Ltd., Beijing (CN)

(72) Inventors: Cheng-Yen Hsieh, Los Angeles, CA (US); Zaixiang ZHENG, Beijing (CN); Yanru QU, Los Angeles, CA (US); Quanquan GU, Los Angeles, CA (US)

(21) Appl. No.: 19/405,733

(22) Filed: Dec. 2, 2025

(51) Int. Cl. G16B 15/20; G16B 40/20

(52) U.S. Cl. CPC G16B 15/20; G16B 40/20

(57) ABSTRACT

Embodiments of the present disclosure provides a method, an electronic device and a storage medium. In the method, a number of residues of a protein structure is obtained, a protein backbone of the protein structure in a first scale is generated, and the protein structure in a second scale is generated based on the number and the protein backbone, where the second scale is larger than the first scale.

### FIELD

Embodiments of the present disclosure mainly relate to the field of biology, and more particularly, to a method, an electronic device and a storage medium.

### BACKGROUND

In the field of biological science research, constructing protein structures with specific sizes and functions may be considered one of the core demands driving life science research and drug development. Traditionally, protein construction has relied on the splicing of fragments of natural sequences. In recent years, breakthrough progress in artificial intelligence technology has provided new solutions for the generation of protein structures.

The application of artificial intelligence in the field of protein structures has evolved much, enabling the creation of novel molecular topologies not found in nature. This provides unprecedented flexibility and efficiency to meet the diverse protein size requirements in bioscience research, and promotes life science research towards a more precise and controllable molecular design stage.

### SUMMARY

Embodiments of the present disclosure provide a method, an electronic device and a storage medium.

In a first aspect of the present disclosure, a method is provided. The method includes: obtaining a number of residues of a protein structure; generating a protein backbone of the protein structure in a first scale; and generating, based on the number and the protein backbone, the protein structure in a second scale, the second scale being larger than the first scale.

In a second aspect of the present disclosure, an electronic device is provided. The electronic device includes: at least one processor; and at least one memory, which is coupled to the at least one processor and stores instructions being executed by the at least one processor. The instruction, when executed by the at least one processor, causes the electronic device to: obtain a number of residues of a protein structure; generate a protein backbone of the protein structure in a first scale; and generate, based on the number and the protein backbone, the protein structure in a second scale, the second scale being larger than the first scale.

In a third aspect of the present disclosure, a non-transitory computer-readable storage medium is provided. The computer-readable storage medium has a machine-executable instruction stored thereon. The machine-executable instruction, when executed by a device, causes the device to perform the method described in the first aspect of the present disclosure.

It is to be understood that the summary section is not intended to identify key or essential features of embodiments of the present disclosure, nor is it intended to be used to limit the scope of the present disclosure. Other features of the present disclosure will become easily comprehensible through the following description.

### BRIEF DESCRIPTION OF THE DRAWINGS

Some example embodiments will now be described with reference to the accompanying drawings, in which:

FIG. 1 illustrates a schematic diagram of an example environment in which embodiments of the present disclosure may be implemented;

FIG. 2 illustrates a flowchart of a method according to an embodiment of the present disclosure;

FIG. 3 illustrates a schematic diagram of an inference process according to an embodiment of the present disclosure;

FIG. 4 illustrates a schematic diagram of an iterative process according to an embodiment of the present disclosure;

FIG. 5 illustrates a schematic diagram of generating a protein structure based on a specific protein backbone according to an embodiment of the present disclosure;

FIG. 6 illustrates a schematic diagram of the effect of generating protein structures based on specific protein backbones according to an embodiment of the present disclosure;

FIG. 7 illustrates a schematic diagram of a motif-based protein structure according to an embodiment of the present disclosure;

FIG. 8 illustrates a schematic diagram of the effect of generating a carrier for a motif according to an embodiment of the present disclosure;

FIG. 9 illustrates a schematic diagram of training a model according to an embodiment of the present disclosure; and

FIG. 10 illustrates a schematic block diagram of an example device that can be used to implement embodiments of the present disclosure.

Throughout the drawings, the same or similar reference numerals represent the same or similar elements, unless otherwise indicated.

### DETAILED DESCRIPTION OF EMBODIMENTS

Principle of the present disclosure will now be described with reference to some example embodiments. It is to be understood that these embodiments are described only for the purpose of illustration and help those skilled in the art to understand and implement the present disclosure, without suggesting any limitation as to the scope of the disclosure. The disclosure described herein can be implemented in various manners other than the ones described below.

In the following description and claims, unless defined otherwise, all technical and scientific terms used herein have the same meaning as commonly understood by one of ordinary skills in the art to which this disclosure belongs.

References in the present disclosure to “one embodiment,” “an embodiment,” “an example embodiment,” and the like indicate that the embodiment described may include a particular feature, structure, or characteristic, but it is not necessary that every embodiment includes the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

It shall be understood that although the terms “first” and “second” etc. may be used herein to describe various elements, these elements should not be limited by these terms. These terms are only used to distinguish one element from another. For example, a first element could be termed a second element, and similarly, a second element could be termed a first element, without departing from the scope of example embodiments. As used herein, the term “and/or” includes any and all combinations of one or more of the listed terms.

The terminology used herein is for the purpose of describing particular embodiments only and is not intended to be limiting of example embodiments. As used herein, the singular forms “a”, “an” and “the” are intended to include the plural forms as well, unless the context clearly indicates otherwise. It will be further understood that the terms “comprises”, “comprising”, “has”, “having”, “includes” and/or “including”, when used herein, specify the presence of stated features, elements, and/or components etc., but do not preclude the presence or addition of one or more other features, elements, components and/or combinations thereof. As used herein, “at least one of the following: <a list of two or more elements>” and “at least one of <a list of two or more elements>” and similar wording, where the list of two or more elements are joined by “and” or “or”, mean at least any one of the elements, or at least any two or more of the elements, or at least all the elements.

In related arts, simple autoregressive models can be used to predict protein structures. These simple autoregressive models employ next-token prediction, predicting the probability of each token based on previous tokens. However, the protein structures generated this way are often unreasonable, similar to the training data and yield poor results.

The inventors of the present application have found that such autoregressive models rely on data discretization for prediction, which compromises the rationality and fine-grained details of the protein structure. Furthermore, protein residues exhibit significant bidirectional dependencies: residues far apart in sequence may be spatially close and form hydrogen bonds or hydrophobic contacts. This bidirectional dependency conflicts with the unidirectional assumption of simple autoregressive models, thus reducing the quality of the predicted protein structures. In short, when predicting the coordinates of one residue, these autoregressive models cannot account for the influence of subsequent residues' positions, leading to unreasonable or inaccurate generated protein structures.

Embodiments of the present disclosure provide a method, an electronic device and a storage medium. The method, electronic device and storage medium involve generating a smaller-scale protein backbone representing the general profile of the protein structure, and then generating the full-scale protein structure based on the desired number of residues in the protein and the protein backbone. By refining the profile of the protein structure, this method can consider the bidirectional dependencies between residues during the generation process, thereby improving the accuracy, rationality, and diversity of the protein structures.

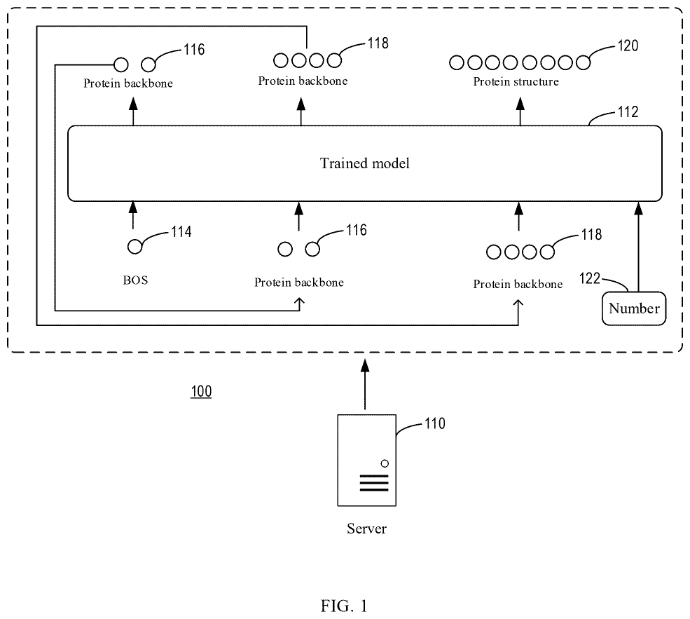

The following will further describe embodiments of the present disclosure in detail with reference to the accompanying drawings. FIG. 1 shows a schematic diagram of an example environment 100 in which embodiments of the present disclosure can be implemented. The example environment 100 includes a server 110. A trained model 112 (e.g., a transformer, a multimodal model capable of processing multimodal data, a decoder, and combinations thereof, etc.) can be deployed on the server 110. In this embodiment, the method of the embodiment of the present disclosure is executed by the server 110.

In some embodiments, a number of residues of a protein structure can be obtained by the server 110. The number of residues may be a user-input number 122. For example, a user may desire to generate a protein structure containing 100 residues. The value 100 representing the number 122 can be input into the trained model 112. In some embodiments, the number of residues 122 can be determined automatically by the model. In some embodiments, one option can be selected by the user from options for a plurality of numbers as a number of residues for the protein structure.

In some embodiments, the protein backbone of the protein structure in a first scale can be generated by the server 110 using the trained model 112. For example, the protein backbone 116 in the first scale can be generated by the trained model 112 based on a BOS (Begin of Sentence) feature 114. The BOS feature 114 is a learnable feature that is determined during the training process. This protein backbone 116 in the first scale represents the profile of the protein structure in the first scale. For instance, the protein backbone 116 in the first scale can be represented by the coordinates of 16 points in three-dimensional space, and the general profile of the protein structure can be indicated by the coordinates of these 16 points. In some embodiments, the protein backbone 116 in the first scale can be input into the trained model 112. Based on the BOS feature 114 and the protein backbone 116 in the first scale, a protein backbone 118 in an intermediate scale is then generated by the trained model 112. This intermediate scale is larger than the first scale. For example, the protein backbone 118 in the intermediate scale can include coordinates for 32 points. In this way, the profile of the protein structure can be refined step by step. In some embodiments, iteration can be continued as described above—the protein backbone 118 in the intermediate scale can be input into the trained model 112 to generate protein backbones in other intermediate scales, thereby progressively refining the profile of the protein structure. For convenience of description, only one intermediate-scale protein backbone is shown in this embodiment. In some embodiments, the protein structure 120 can be generated directly by the server 110 based on the protein backbone 116 in the first scale, without generating protein backbone 118 in the intermediate scale. To facilitate describing the core principles, excessive network layers are not introduced in this embodiment of the model, but the methods of the embodiments of the present disclosure can be applied by those skilled in the art to various models with different architectures.

In some embodiments, the protein structure 120 in a second scale can be generated by the server 110 based on the number and the protein backbone(s). That is, the protein structure 120 in the second scale can be generated by the server 110 based on the user-input number 122 (e.g., 100), the protein backbone 116 in the first scale, and the protein backbone 118 in the intermediate scale. This second scale is larger than the first and intermediate scales. The protein structure 120 in the second scale can be three-dimensional coordinates for 100 points. In some embodiments, each point can be used to represent a carbon α atom in the protein structure, and the coordinates of the carbon α atom are represented by the coordinates of the point.

In this embodiment, the multi-scale, iterative generation strategy is utilized, which effectively ensures both the global rationality and local accuracy of the final generated protein structure. A protein backbone represented by only a small number of points (e.g., 16) is first generated by the trained model 112. This enables the overall topology of the protein to be quickly determined, and is then iteratively refined, whereby protein backbones at progressively higher resolutions (e.g., 32 points, 64 points, etc.) are generated. The interactions (including bidirectional and long-range interactions) among all residues are naturally considered, and this global constraint is propagated to subsequent refinement steps. The unidirectional limitation of autoregressive models is fundamentally overcome. The bidirectional dependencies among protein residues can be comprehensively considered, thereby enabling the efficient and reliable generation of three-dimensional protein structures that are structurally reasonable and accurate in detail.

It should be understood that an instance of the server 110 may be an independent physical server, a server cluster or distributed system composed of multiple physical servers, or a cloud server. Basic cloud computing services such as cloud services, cloud databases, cloud computing, cloud functions, cloud storage, network services, cloud communication, middleware services, domain name services, security services, CDN, as well as big data and artificial intelligence platforms are provided by such a cloud server. Connections between servers can be made directly or indirectly via wired or wireless communication means, which is not limited herein.

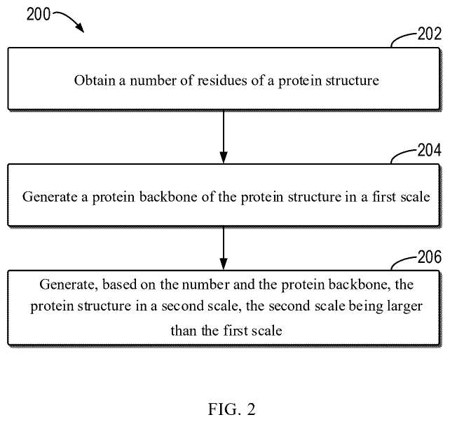

FIG. 2 shows a flowchart of a method 200 according to some embodiments of the present disclosure. In this embodiment, the method can be executed by the server 110 of the embodiment in FIG. 1. At block 202, a number of residues of a protein structure is obtained. A protein structure is composed of polypeptide chains and has different structural hierarchies, such as primary structure, secondary structure, tertiary structure, and quaternary structure. Embodiments of the present disclosure can be used to generate protein structures belonging to one or more of these primary, secondary, tertiary, and quaternary structures. In a protein sequence, after each amino acid forms a peptide chain, it loses some atoms, and the remaining part is called a residue. The number refers to the total number of residues that the user wishes the generated protein structure to contain, which determines the scale of the final generated protein structure. For example, a user may desire to design a new small protein, such as a protein structure containing 100 amino acids. In this operation, a number obtained by the model is 100. This number informs the model that it ultimately needs to generate a protein structure composed of 100 points (each point representing one residue).

At block 204, a protein backbone of the protein structure in a first scale is generated. The protein backbone indicates the profile of the protein structure. The protein backbone is not represented by the residues of the protein structure but is abstractly represented using a small number of key points. The scale represents the resolution, for example, it can be the number of points used to represent the protein backbone. The first scale represents a first resolution. For instance, in a protein backbone comprising 16 points, points 1 to 4 might represent the trajectory of a first alpha helix, points 5 to 8 might represent a loop region, and points 9 to 16 might represent the trajectory of a beta-sheet. The profile of these 16 points is very coarse, but it captures the global topological information of the protein structure: first a helix, then a turn, followed by a sheet formation.

At block 206, the protein structure in a second scale is generated based on the number and the protein backbone, the second scale being larger than the first scale. The second scale may represent the full scale. At this scale, the protein structure is represented in detail, where each point represents the position of an atom or a residue in the protein structure. In this operation, the generated low-resolution profile (first-scale protein backbone) and the known target length (number of residues) are used as constraints to generate the complete protein structure. The final output protein structure in the second scale can be a one-dimensional, two-dimensional, or three-dimensional coordinate set containing 100 points.

This method involves generating a smaller-scale protein backbone representing the general profile of the protein structure, and then generating the full-scale protein structure based on the desired number of residues in the protein and the protein backbone. By refining the profile of the protein structure, this method can consider the bidirectional dependencies between residues during the generation process, thereby improving the accuracy, rationality, and diversity of the protein structures.

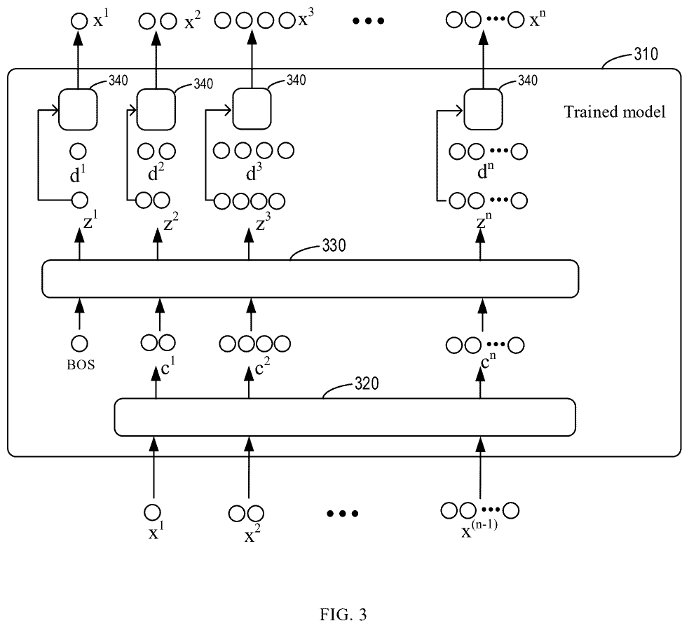

FIG. 3 shows a schematic diagram of an inference process according to an embodiment of the present disclosure. In the present disclosure, the inference process includes multiple iterative processes. In some embodiments, a trained model 310 includes an upsampling module 320, a transformer 330, and a decoder 340. In some embodiments, the decoder 340 can perform decoding through a diffusion process. In FIG. 3, the decoder 340 is shown as multiple decoders 340 to represent multiple diffusion processes, rather than to indicate that the trained model 310 includes multiple decoders. In some embodiments, the trained model 310 includes one decoder 340.

In this embodiment, as an example, the obtained number of residues for the protein structure is 256. In some embodiments, after a BOS feature is input into the transformer 330, a guidance feature z¹ (i.e., the first guidance feature) is generated. This guidance feature z¹ is input into the decoder 340. In some embodiments, a protein backbone x¹ in a first scale is generated by the decoder 340 based on a noise feature d¹ and the guidance feature z¹. The information source for the guidance feature comes from all previously generated, coarser-scale protein backbones. These coarse scales have already outlined the overall shape of the protein, such as whether it is a globular protein or an elongated chain protein, roughly how many domains it has, and whether it is primarily composed of alpha-helices or beta-sheets, etc. The guidance feature encodes information about the global topology. It can provide constraints to the decoder, indicating that the spatial characteristics of the protein structure or protein backbone should be consistent with this global topology.

In some embodiments, one or more intermediate protein backbones in one or more intermediate scales are determined based on the protein structure in the first scale. That is, during the process of generating the protein result, not only can the protein backbone in the first scale be generated, but also, based on the protein backbone in the first scale, one or more (e.g., two) protein backbones in one or more intermediate scales can be generated. For example, the protein backbone x¹ in the first scale generated in the first iterative process includes 16 points, the protein backbone x² in an intermediate scale generated in the second iterative process includes 32 points, the protein backbone x³ in an intermediate scale generated in the third iterative process includes 64 points, and the protein backbone x⁴ in an intermediate scale generated in the fourth iterative process includes 128 points, and so on.

In some embodiments, the protein structure is generated based on the protein backbone in the first scale, the one or more intermediate protein backbones, the number, and the second noise feature. After generating multiple protein backbones, these backbones can be referenced to generate the final protein structure. For example, the protein structure can be generated according to formula (1).

$$p ( x ) = {E}_{X ∼ {q}_{decompose} ( \cdot \vert X )} p ( X = \{ {x}^{1} , … , {x}^{n} \} ) = {E}_{X ∼ {q}_{decompose} ( \cdot \vert X )} {\prod }_{i = 1}^{n} p ( {x}^{i} \vert {X}^{< i} ) \qquad ( 1 )$$

In this formula, x¹ to xⁿ represent the 1st to the nth protein backbone, n is a positive integer greater than 1 and represents the number of scales, i is a positive integer and represents the scale index, E denotes the expected value, x represents the protein structure, the protein backbone xⁿ is equal to the protein structure x, xⁱ represents the protein backbone in the i-th scale, p (x) represents the probability of generating the protein structure using the model, X represents the set of protein backbones, X˜q_{decompose}(⋅|x) represents the autoregressive decomposition of the protein structure x into n scales X={x¹, . . . , xⁿ}, and p (xⁱ|X^{<i}) represents the probability of the model generating the i-th protein backbone.

In the final iterative process used for generating the protein structure, the scale can be flexibly adjusted according to the number. For example, if the obtained number is 200, after the intermediate-scale protein backbone x⁴ comprising 128 points is generated in the fourth iterative process, the upsampling module 320 is used to upsample the protein backbone x⁴, resulting in an upsampled feature at a scale comprising 200 points. A guidance feature is generated based on this upsampled feature, all previously generated upsampled features, and the BOS feature. This guidance feature and a second noise feature are input into the decoder to generate the protein structure. For example, the guidance feature can be calculated according to formula (2).

$${z}^{i} = 𝒯 ( {X}^{< i} ) = 𝒯 ( [ bos , Up ( {x}^{1} , size ( 2 ) ) , … , Up ( {x}^{i - 1} , size ( i ) ) ] ) \qquad ( 2 )$$

In this formula, i is a positive integer, zⁱ represents the i-th guidance feature, [symbol] represents the transformer, Up (xⁱ⁻¹,size(i)) represents upsampling the protein backbone xⁱ⁻¹ to an upsampled feature of scale=i, and X^{<i} represents the set of the 1st to the (i−1)-th protein backbones.

In some embodiments, after the protein backbone x¹ in the first scale is generated, the protein backbone x¹ is input into the upsampling module 320. The protein backbone x¹ is upsampled by the upsampling module 320 into an upsampled feature c¹. The scale of this upsampled feature c¹ can be, for example, twice that of the protein backbone x¹ in the first scale. In this embodiment, through an interpolation algorithm (such as linear interpolation), the upsampling operation determines where the newly added intermediate points should be located based on the coarse-scale points. This effectively constructs a low-resolution contour that is globally structurally largely correct.

In some embodiments, this upsampled feature c¹ is input into the transformer 330 to generate a guidance feature z². This guidance feature z² is input into the decoder 340, where a protein backbone x² is generated by the decoder 340 based on a noise feature d² and the guidance feature z².

In some embodiments, after the protein backbone x² is generated, the protein backbone x² is input into the upsampling module 320. The protein backbone x² is upsampled by the upsampling module 320 into an upsampled feature c². The scale of this upsampled feature c² can be, for example, twice the scale of the protein backbone x².

In some embodiments, this upsampled feature c² is input into the transformer 330. A guidance feature z³ is generated. This guidance feature z³ is input into the decoder 340, where a protein backbone x³ is generated by the decoder 340 based on a noise feature d³ and the guidance feature z³. Iteration can continue in this manner (e.g., n−1 iterations), allowing the protein backbone to be progressively refined. For example, protein backbone x¹ includes 3D coordinates for 16 points, protein backbone x² includes 3D coordinates for 32 points, and protein backbone x³ includes 3D coordinates for 64 points. Through one or more iterative processes, the number of points included in the protein backbone approaches the desired number for the protein.

In some embodiments, in the n-th iteration, the protein backbone x(n−1) is input into the upsampling module 320 to obtain an upsampled feature cⁿ. In some embodiments, this upsampled feature cⁿ is input into the transformer 330 to obtain a guidance feature zⁿ (i.e., the second guidance feature in the second scale). This guidance feature zⁿ is input into the decoder 340. The protein structure xⁿ is generated by the decoder 340 based on a noise feature dⁿ (i.e., the second noise feature) and the guidance feature zⁿ. In some embodiments, this protein structure xⁿ includes 3D coordinates for 256 points, i.e., the 3D coordinates of 256 carbon α atoms. It should be understood that n can be any positive integer greater than or equal to 2, depending on the specific situation. When n=2, the trained model 310 can generate the protein structure xⁿ after being configured for two iterations.

In some embodiments, a denoising vector is determined at the decoder 340 based on the guiding feature zⁿ and the noise feature dⁿ, wherein the denoising vector indicates a denoising direction and a denoising speed. The noise feature is a representation of the protein structure in its current state. At the beginning of the diffusion process, it can be pure random noise. As denoising proceeds, it gradually incorporates more and more real structural information. This denoising vector is the vector used to restore the noise feature into the protein backbone. Starting from the noise feature, the denoising vector points towards the final protein backbone with a specific direction and magnitude. To improve the accuracy of the diffusion process, the denoising vector can be continuously updated through iterative operations within the decoder.

In some embodiments, the following operations are iteratively performed until a preset stopping condition is met: the denoised second noise feature is determined by denoising the second noise feature according to the denoising vector, and the denoising vector is updated based on the second guiding feature and the denoised second noise feature. In this iterative operation, the denoising process of the noise feature is divided into multiple stages, with the noise feature being partially denoised each time, which can make the restoration of the noise feature more accurate. Each time the denoising vector is updated, the guiding feature is referenced, ensuring that the generated protein backbone is reasonable and accurate.

In some embodiments, the denoised second noise feature is taken as the protein structure, wherein the denoised second noise feature comprises three-dimensional coordinates of a plurality of carbon α atoms. By continuously denoising the noise feature, the protein structure can be generated, represented by the three-dimensional coordinates of the multiple carbon α atoms.

In some cases, factors such as overfitting can reduce the accuracy of the generated protein backbones and protein structures. To address this, small random noise can be added to intermediate products. In some embodiments, during the denoising process of determining the denoised second noise feature by denoising the second noise feature according to the denoising vector, an initial noise feature is first determined based on the denoising vector and the second noise feature. That is, denoising is first performed to a certain extent according to the denoising vector. In some embodiments, the denoised second noise feature is then determined based on the initial noise feature and random noise. After denoising, a small amount of random noise is added to the initial noise feature, which can mitigate the negative effects caused by factors like overfitting and exposure bias.

In some embodiments, both the first noise feature and the second noise feature belong to Gaussian noise. A dimension of the first noise feature is the same as a dimension of the protein backbone, and a dimension of the second noise feature is the same as a dimension of the protein structure. Gaussian noise refers to random noise that follows a Gaussian distribution (i.e., a normal distribution). In three-dimensional space, this means the noise can randomly offset in any direction with varying magnitudes, but most offsets will be concentrated near the mean value. The Gaussian distribution has very favorable mathematical properties, such as additivity and stability, which ensure that both the forward noising and reverse denoising steps of the diffusion process are solvable, making the training and inference processes more stable and efficient.

The dimension of the first noise feature is the same as the dimension of the protein backbone, so that the first noise feature in the first scale can be used to generate the protein backbone in the first scale. The dimension of the second noise feature is the same as the dimension of the protein structure, so that the second noise feature in the second scale can be used to generate the protein structure in the second scale.

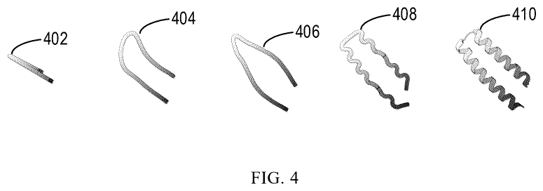

FIG. 4 shows a schematic diagram of an iterative process according to an embodiment of the present disclosure. As shown in FIG. 4, the protein backbone x¹ output by the first iterative process is shown as profile 402, the protein backbone x² output by the second iterative process is shown as profile 404, the protein backbone x³ output by the third iterative process is shown as profile 406, the protein backbone x⁴ output by the fourth iterative process is shown as profile 408, and the protein backbone x⁵ output by the fifth iterative process is shown as profile 410. It can be seen from FIG. 4 that the profile of the protein structure is progressively refined, with increasing detail as the iterative process proceeds, ultimately resulting in a relatively reasonable protein structure.

In each iterative process, the scale size is continuously increased. The specific magnitude of increase can be determined based on the magnitude change during training. For example, if during training the upsampling module of the model increases the scale of the input data to twice its original size, then during the iterative process, the upsampling module will still increase the scale of the input data to twice its size, ensuring relatively accurate predictive capability. Of course, in the final iterative process, as mentioned above, the scale needs to be adjusted according to the number of residues of the desired protein structure, i.e., the parameters of the upsampling module are adjusted to generate an upsampled feature with a dimension corresponding to that number.

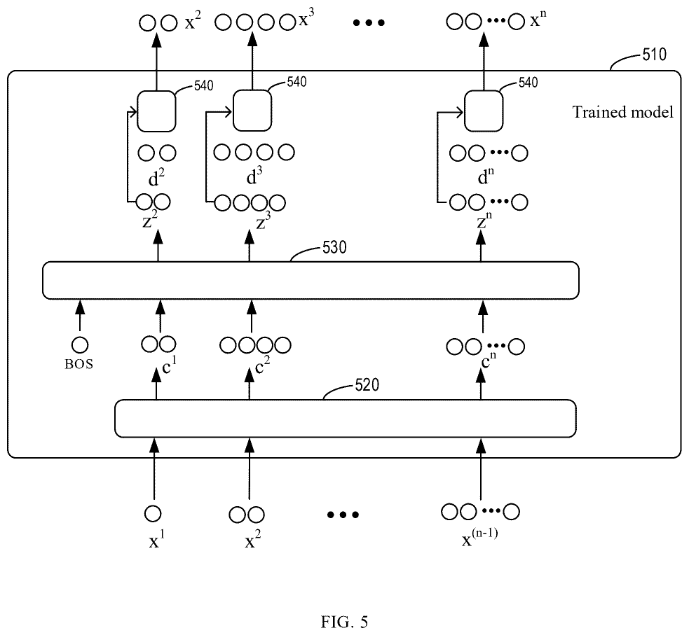

The model can not only generate a protein structure starting from a BOS feature but can also generate a protein structure based on a specific protein backbone. FIG. 5 shows a schematic diagram of generating a protein structure based on a specific protein backbone according to an embodiment of the present disclosure. In some embodiments, a protein backbone x¹ in a third scale (i.e., a third protein backbone) is obtained. For example, this protein backbone x¹ can be a protein backbone provided by a user.

In some embodiments, a protein structure xⁿ (i.e., a second protein structure) is generated based on the protein backbone x¹. For example, at the beginning of inference, the protein backbone x¹ can be input into an upsampling layer 520 to obtain an upsampled feature c¹, rather than starting from the BOS feature. After generating the upsampled feature c¹, the BOS feature and the upsampled feature c¹ are input together into a transformer 530 to generate a guidance feature z². The guidance feature z² and a noise feature d² are then input together into a decoder 540 to obtain a protein backbone x². This completes the first iteration. Multiple iterations are performed in a manner similar to the above-described embodiment, thereby generating the protein structure xⁿ.

In related arts, models can only generate protein structures similar to the training data and cannot consider the bidirectional dependencies between residues. In this embodiment, an accurate protein structure can be generated based on a preset protein backbone, thereby improving the diversity and rationality of the generated protein structures.

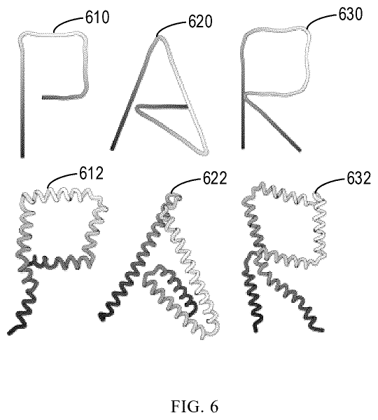

FIG. 6 shows a schematic diagram of the effect of generating protein structures based on specific protein backbones according to an embodiment of the present disclosure. In this embodiment, the protein backbones provided by the user can be protein backbone 610, protein backbone 620, and protein backbone 630 (i.e., contours roughly resembling the letters P, A, R). These protein backbones can be used as starting points for the inference process to generate protein structures respectively. The lower part of FIG. 6 shows the generated protein structure 612, protein structure 622, and protein structure 632, respectively. As can be seen from FIG. 6, selecting different protein backbones as the profile results in significantly different protein structures. The protein structures 612, 622, and 632 all essentially follow the initially set contours, i.e., the letters P, A, R. It can also be observed that the guidance feature has a relatively strong conditioning ability in preserving contour information, thereby ensuring that the generated protein structures have relatively reasonable structures and can preserve the bidirectional dependency information between residues.

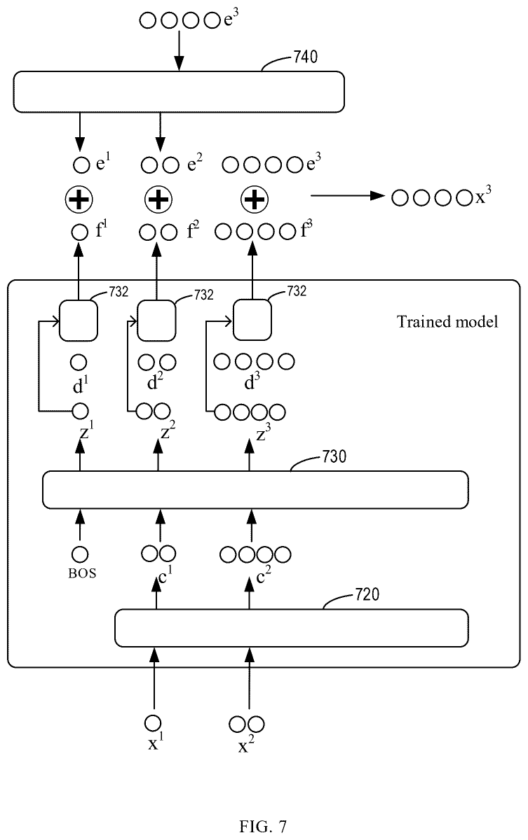

In another scenario, the model can be used to design motif-based proteins. FIG. 7 shows a schematic diagram of a motif-based protein structure according to an embodiment of the present disclosure. In some embodiments, a protein structure e³ (i.e., a third protein structure) is obtained, wherein the protein structure e³ includes a first portion (i.e., the motif portion). Another portion of the protein structure e³ can be, for example, a carrier portion. This protein structure e³ can be, for example, a protein structure provided by a user.

In some embodiments, a downsampling module 740 is used to downsample the protein structure e³ to determine a plurality of reference backbones, such as reference backbone e¹ and reference backbone e². These reference backbones respectively record the accurate coordinates of the points representing the motif in different scales.

In some embodiments, an edited first protein backbone is determined by replacing a partial backbone of the protein backbone in the first scale corresponding to the first portion with a partial backbone of the reference backbone in the first scale corresponding to the first portion. For example, in some embodiments, after a BOS feature is input into a transformer 730, a guidance feature z¹ (i.e., a first guidance feature) is generated. This first guidance feature z¹ is input into a decoder 732. In some embodiments, a protein backbone f1 in the first scale is generated by the decoder 732 based on a noise feature d′ and the guidance feature z¹. As an example, the protein backbone f1 includes 3D coordinates for 16 points, where points 15 and 16 represent the profile of the first portion. The reference backbone e¹ also includes 3D coordinates for 16 points, where points 15 and 16 represent the profile of the first portion. At this point, the data for points 15 and 16 from the reference backbone e¹ can be used to replace points 15 and 16 in the protein backbone f1, resulting in an edited first protein backbone x¹.

In some embodiments, a protein structure x³ (i.e., a fourth protein structure) is generated based on the edited first protein backbone x¹. For example, in some embodiments, the edited first protein backbone x¹ is input into an upsampling module 720 to obtain an upsampled feature c¹. Then, the BOS feature and the feature c¹ are input into the transformer 730 to generate a guidance feature z². The guidance feature z² and a noise feature d² are input into the decoder 732 to obtain a protein backbone f2 in a third scale. Assume both the reference backbone e² and the protein backbone f2 have a scale of 32 points. Points 29-32 of the reference backbone e² reflect the true profile of the motif, while points 1-28 of the protein backbone f2 reflect the profile of the carrier outside the motif, and points 29-32 reflect the predicted profile of the motif. At this point, points 29-32 of the reference backbone e² can be used to replace points 29-32 in the protein backbone f2, resulting in an edited protein backbone x² in the third scale.

In some embodiments, a protein structure f3 in a second scale is generated based on the edited first protein backbone x¹ and the edited second protein backbone x². In a similar manner, the portion of the protein structure e³ corresponding to the motif is used to replace the portion of the protein structure f3 corresponding to the motif, resulting in the protein structure x³. This allows for the generation of different protein structures containing the same motif, where the protein structure considers the bidirectional dependencies between residues and has high rationality and accuracy.

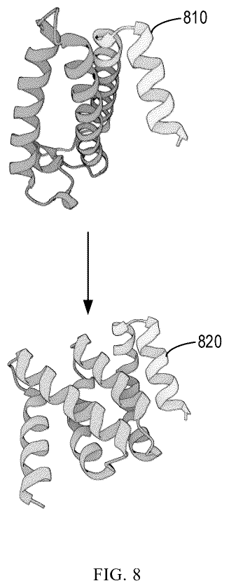

FIG. 8 shows a schematic diagram of the effect of generating a carrier for a motif according to an embodiment of the present disclosure. As shown in FIG. 8, the protein structure e³ can be protein structure 810, where the peptide chain on the far right is the motif, and the other parts are the carrier for this motif. After processing according to the above embodiment, protein structure 820 can be generated. Comparing protein structure 810 and protein structure 820, they have the same motif but different carriers, demonstrating that embodiments of the present disclosure can provide diverse and accurate carriers for a motif.

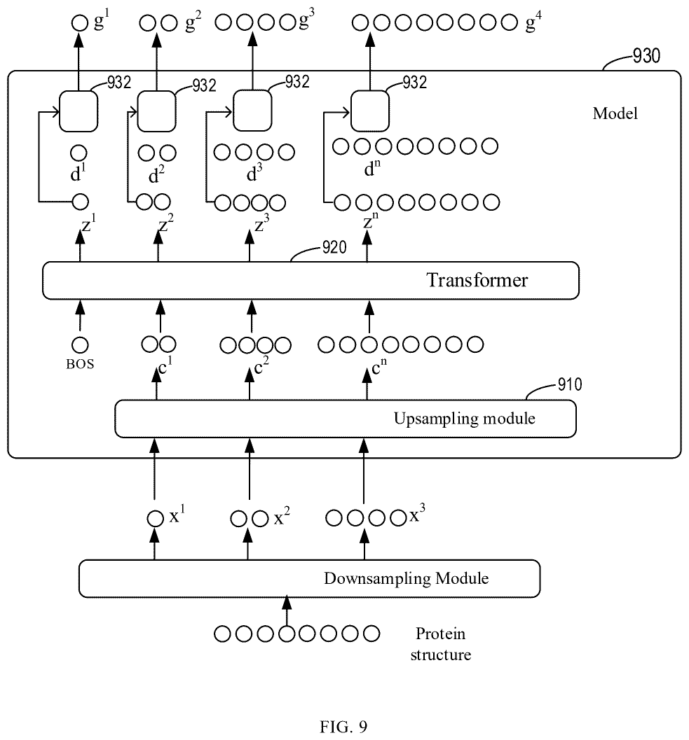

FIG. 9 shows a schematic diagram of training a model according to an embodiment of the present disclosure. In some embodiments, a protein structure sample is obtained. This protein structure sample possesses relatively accurate spatial information of the protein structure. For example, the protein structure sample can be derived from natural protein structures, or so-called real protein structures.

In some embodiments, a plurality of reference backbones in a plurality of scales are determined by downsampling the protein structure sample. These scales are different from each other in size. For example, the scales may include 3 scales, comprising 5 points, 10 points, and 20 points, respectively. Because the protein structure sample has accurate spatial information, the plurality of reference backbones in the plurality of scales can be used as labels for subsequent training. The plurality of reference backbones in the plurality of scales can be reference backbone x¹, reference backbone x², and reference backbone x³.

In some embodiments, the model 930 is trained based on the plurality of reference backbones and the protein structure sample. Among the plurality of reference backbones, a reference backbone in a smaller scale can be used as input data, and the model 930 generates output data based on this reference backbone in a smaller-scale. A reference backbone in a larger scale can be used as a label for the output data, thereby calculating a loss, and the parameters of the model 930 are updated based on the loss.

In some embodiments, a plurality of upsampled features for training are determined by upsampling the plurality of reference backbones. As shown in FIG. 9, to improve training efficiency, batch data can be used for training. For example, the reference backbone x¹, reference backbone x², and reference backbone x³ are all input into an upsampling module 910, resulting in upsampled feature c¹, upsampled feature c², and upsampled feature c³. The upsampled feature c¹, upsampled feature c², upsampled feature c³, and a BOS feature are input into a transformer 920 to obtain guidance feature z¹, guidance feature z², guidance feature z³, and guidance feature z⁴. The guidance feature z¹ is determined by the transformer 920 based on the BOS feature; the guidance feature z² is determined by the transformer 920 based on the BOS feature and the upsampled feature c¹; the guidance feature z³ is determined by the transformer 920 based on the BOS feature, the upsampled feature c¹, and the upsampled feature c²; and the guidance feature z⁴ is determined by the transformer 920 based on the BOS feature, the upsampled feature c¹, the upsampled feature c², and the upsampled feature c³.

In some embodiments, one or more predicted protein backbones and a predicted protein structure are generated based on the plurality of upsampled features for training and a plurality of noise features for training. The decoder 932 is replicated into 4 decoders 932. The guidance feature z¹ and a noise feature d¹ are input into a decoder 932; the guidance feature z² and a noise feature d² are input into a decoder 932; the guidance feature z³ and a noise feature d³ are input into a decoder 932; and the guidance feature z⁴ and a noise feature d⁴ are input into a decoder 932, to obtain the predicted protein backbone g¹, the predicted protein backbone g², the predicted protein backbone g³, and the predicted protein structure g⁴.

When training is performed using natural protein structures, since the inference process is often used to generate protein structures not yet found in nature, this discrepancy can lead to exposure bias. Exposure bias refers to the phenomenon where the input distribution encountered by the model 930 during the training phase differs from that during the inference phase, causing performance degradation of the trained model 930 during actual generation.

In some embodiments, the plurality of noise features for training are determined based on the plurality of reference backbones, the protein structure sample, and a plurality of Gaussian noises. For example, the noise features for training can be determined according to formula (3).

$${x}_{{t}^{i}}^{i} = {t}^{i} \cdot {x}^{i} + ( 1 - {t}^{i} ) \cdot {ϵ}^{i} \qquad ( 3 )$$

In this formula, tⁱ represents a randomly sampled timestep in the range [0,1], ϵⁱ represents Gaussian noise, i is a positive integer representing the scale index, and xⁱ represents the protein backbone at the i-th scale. In some embodiments, the parameters of the model 930 are updated based on the loss. For example, backpropagation and gradient update can be used to update the parameters of the model 930. During the update process, the BOS feature can also be updated. This allows the model 930 to learn to work in imperfect contexts, thereby reducing the impact of exposure bias.

In some embodiments, the loss is determined based on the plurality of reference backbones, the protein structure sample, the one or more predicted protein backbones, and the predicted protein structure. For example, the loss can be calculated according to formula (4).

$$ℒ = {E}_{x} [ \frac{1}{n} {\sum }_{i = 1}^{n} \frac{1}{size ( i )} {E}_{{t}^{i} ∼ p ( i ) , {ϵ}^{i} ∼ N ( 0 , I )} [ {\vert v ( {x}_{t}^{i} , {t}^{i} , zi ) - ( {x}^{i} - {ϵ}^{i} ) \vert}^{2} ] ] \qquad ( 4 )$$

In this formula, [symbol] represents the loss of the model 930 (which at least includes the transformer and the decoder), i is a positive integer representing the scale index, size(i) represents the size of the i-th scale, n is the number of scales, x represents the protein structure sample, xⁱ represents the reference backbone at the i-th scale, ϵⁱ represents Gaussian noise, zⁱ represents the i-th guidance feature, tⁱ represents a randomly sampled timestep in the range [0,1],

$${x}_{t}^{i}$$

represents the noise feature, p(i) represents the sampling distribution for timestep tⁱ, E denotes the expected value, N(0,I) represents a multivariate Gaussian distribution with mean 0 and covariance matrix I (identity matrix), and

$$v ( {x}_{t}^{i} , {t}^{i} , zi )$$

represents the output data of the decoder given the input guidance feature zⁱ, timestep tⁱ, and noise feature

$${x}_{t}^{i} .$$

To prevent overfitting during training and improve training effectiveness, in some embodiments, a plurality of adjusted reference backbones are determined by adding noise to the plurality of reference backbones. For example, each adjusted reference backbone can be determined by calculating a weighted sum of each reference backbone and Gaussian noise. For instance, the adjusted reference backbone can be calculated according to formula (5).

$${x}^{{i}^{′}} = a \times {x}^{i} + ( 1 - a ) \cdot {ϵ}^{i} \qquad ( 5 )$$

In this formula, i is a positive integer representing the scale index, xⁱ represents the protein backbone at the i-th scale, a represents a weight coefficient, ϵⁱ represents Gaussian noise, and xⁱ′ represents the adjusted reference backbone. The weight coefficient a can be, for example, a preset weight coefficient.

In some embodiments, the plurality of upsampled features for training are determined by upsampling the plurality of adjusted reference backbones. This introduces a certain amount of noise into the upsampled features used for training, and the protein backbone or protein structure is predicted based on these upsampled features for training. This helps train the model's error correction capability, thereby improving the training effectiveness of the model.

During training, the process can be conducted according to preset scales. For example, the first scale can be preset to a first value, an intermediate scale can be preset to a second value, and the second scale can be preset to a third value. Alternatively, assuming the desired length of the protein structure is L, the first scale can be preset to L/4, the intermediate scale to L/2, and the second scale to L. Thus, during inference, the model will prioritize inference according to the scales preset during training until a protein structure of length L is generated.

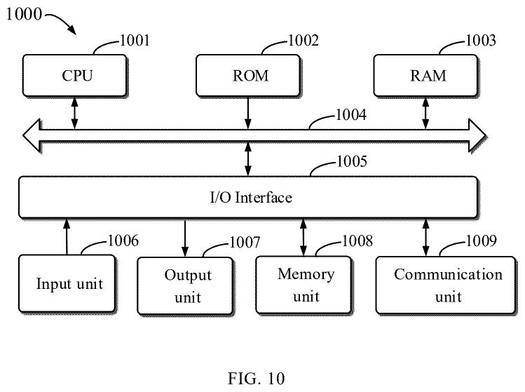

FIG. 10 illustrates a simplified block diagram of a device 1000 that is suitable for implementing some example embodiments of the present disclosure. As illustrated therein, the device 1000 includes a central processing unit (CPU) 1001 that may perform various appropriate actions and processing based on computer program instructions stored in a Read-Only Memory (ROM) 1002 or loaded from a memory unit 1008 to a Random-Access Memory (RAM) 1003. In the RAM 1003, there may further store various programs and data needed for operations of the device 1000. The CPU 1001, ROM 1002 and RAM 1003 are connected to each other via a bus 1004. An input/output (I/O) interface 1005 is also connected to the bus 1004.

Various components in the device 1000 are connected to the I/O interface 1005, including: an input unit 1006 such as a keyboard, a mouse and the like; an output unit 1007 such as various types of displays and loudspeakers, etc.; a memory unit 1008 such as a magnetic disk, an optical disk, and etc.; and a communication unit 1009 such as a network card, a modem, and a wireless communication transceiver, etc. The communication unit 1009 allows the device 1000 to exchange information/data with other devices via a computer network such as the Internet and/or various types of telecommunications networks. It is understood that the present disclosure may display, via the output unit 1007, real-time dynamic change information of the customer satisfaction, key factor identification information of a group of customers or individual customers subjected to the satisfaction, optimized strategy information, and strategy implementation effect assessment information, etc.

The processing unit 1001 may be implemented by one or more processing circuits. The processing unit 1001 may be configured to perform various processes and processing described above. For example, in some embodiments, the process described above may be implemented as a computer software program that is tangibly embodied on a machine readable medium, e.g., the memory unit 1008. In some embodiments, part or all of the computer program may be loaded and/or mounted onto the device 1000 via ROM 1002 and/or communication unit 1009. When the computer program is loaded to the RAM 1003 and executed by the CPU 1001, one or more steps of the process as described above may be executed.

It is to be understood that although FIG. 10 is illustrated as an illustrative device to perform the process or method illustrated above, the embodiments of the present disclosure may also be implemented at one or more quantum computers, the present disclosure does not limit this aspect.

The present disclosure may be implemented a system, a method and/or a computer program product. The computer program product may comprise a computer-readable storage medium on which computer-readable program instructions for executing various aspects of the present disclosure are loaded.

The computer readable storage medium may be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium comprises the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform various aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processing unit of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the drawings illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It is also to be noted that, in some alternative implementations, the functions noted in the block may occur out of the order noted in the figures. For example, two blocks illustrated in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

In one aspect, there is provided a method, such as a computer-implemented method. The method comprises: obtaining a number of residues of a protein structure, generating a protein backbone of the protein structure in a first scale; and generating, based on the number and the protein backbone, the protein structure in a second scale, the second scale being larger than the first scale.

In some implementations, generating a protein backbone of the protein structure in a first scale comprises: generating a first guiding feature in the first scale; and generating, based on the first guiding feature and a first noise feature, the protein backbone in the first scale.

In some implementations, generating, based on the number and the protein backbone, the protein structure in the second scale comprises: determining, by upsampling the protein backbone, a first upsampled feature in the second scale; generating, based on the first upsampled feature, a second guiding feature in the second scale; and generating, based on the second guiding feature and a second noise feature, the protein structure.

In some implementations, generating, based on the second guiding feature and the second noise feature, the protein structure comprises: determining, based on the second guiding feature and the second noise feature, a denoising vector, wherein the denoising vector indicates a denoising direction and a denoising speed; iteratively performing the following operations until a preset stopping condition is met: determining the denoised second noise feature by denoising the second noise feature according to the denoising vector; and updating, based on the second guiding feature and the denoised second noise feature, the denoising vector; and taking the denoised second noise feature as the protein structure, wherein the denoised second noise feature comprises three-dimensional coordinates of a plurality of carbon α atoms.

In some implementations, determining the denoised second noise feature by denoising the second noise feature according to the denoising vector comprises: determining, based on the denoising vector and the second noise feature, an initial noise feature; and determining, based on the initial noise feature and random noise, the denoised second noise feature.

In some implementations, both the first noise feature and the second noise feature belong to Gaussian noise, a dimension of the first noise feature is the same as a dimension of the protein backbone in the first scale, and a dimension of the second noise feature is the same as a dimension of the protein structure.

In some implementations, generating, based on the number and the protein backbone, the protein structure in the second scale comprises: determining, based on the protein backbone in the first scale, one or more intermediate protein backbones in one or more intermediate scales; and generating the protein structure based on the first protein backbone, the one or more intermediate protein backbones, the number and the second noise feature.

In some implementations, the method further comprises: obtaining a third protein backbone in a third scale; and generating, based on the third protein backbone, a second protein structure.

In some implementations, the method further comprises: obtaining a third protein structure, wherein the third protein structure includes a first portion; determining a plurality of reference backbones by downsampling the third protein structure; determining an edited first protein backbone by replacing a partial backbone of the protein backbone in the first scale corresponding to the first portion with a partial backbone of a reference backbone in the first scale corresponding to the first portion; and generating a fourth protein structure based on the edited first protein backbone.

In some implementations, the method is performed by a model, and the method further comprises: obtaining a protein structure sample; determining a plurality of reference backbones in a plurality of scales by downsampling the protein structure sample; and training the model based on the plurality of reference backbones and the protein structure sample.

In some implementations, training the model based on the plurality of reference backbones and the protein structure sample comprises: determining a plurality of upsampled features for training by upsampling the plurality of reference backbones; generating one or more predicted protein backbones and a predicted protein structure based on the plurality of upsampled features for training, and a plurality of noise features for training; determining a loss based on the plurality of reference backbones, the protein structure sample, the one or more predicted protein backbones and the predicted protein structure; and updating parameters of the model based on the loss.

In some implementations, determining the plurality of upsampled features for training by upsampling the plurality of reference backbones comprises: determining a plurality of adjusted reference backbones by adding noise to the plurality of reference backbones; and determining the plurality of upsampled features for training by upsampling the plurality of adjusted reference backbones.

In some implementations, the plurality of noise features for training are determined based on the plurality of reference backbones, the protein structure sample and a plurality of Gaussian noise.

In another aspect, there is provided an electronic device. The electronic device comprises: at least one display; at least one memory; and at least one processor coupled with the at least one memory and configured to cause the device to: obtain a number of residues of a protein structure; generate a protein backbone of the protein structure in a first scale; and generate, based on the number and the protein backbone, the protein structure in a second scale, the second scale being larger than the first scale.

In some implementations, the electronic device is further caused to: generate a first guiding feature in the first scale; and generate, based on the first guiding feature and a first noise feature, the protein backbone in the first scale.

In some implementations, the electronic device is further caused to: determine, by upsampling the protein backbone, a first upsampled feature in the second scale; generate, based on the first upsampled feature, a second guiding feature in the second scale; and generate, based on the second guiding feature and a second noise feature, the protein structure.

In some implementations, the electronic device is further caused to: determine, based on the second guiding feature and the second noise feature, a denoising vector, wherein the denoising vector indicates a denoising direction and a denoising speed; iteratively perform the following operations until a preset stopping condition is met: determining the denoised second noise feature by denoising the second noise feature according to the denoising vector; and updating, based on the second guiding feature and the denoised second noise feature, the denoising vector; and take the denoised second noise feature as the protein structure, wherein the denoised second noise feature comprises three-dimensional coordinates of a plurality of carbon α atoms.

In some implementations, the electronic device is further caused to: determine, based on the denoising vector and the second noise feature, an initial noise feature; and determine, based on the initial noise feature and random noise, the denoised second noise feature.

In some implementations, both the first noise feature and the second noise feature belong to Gaussian noise, a dimension of the first noise feature is the same as a dimension of the protein backbone in the first scale, and a dimension of the second noise feature is the same as a dimension of the protein structure.

In a further aspect, there is provided a non-transitory computer-readable storage medium having instructions stored thereon, the instructions, when executed by a processor of a device, causing the device to: obtain a number of residues of a protein structure; generate a protein backbone of the protein structure in a first scale; and generate, based on the number and the protein backbone, the protein structure in a second scale, the second scale being larger than the first scale.

Although the present disclosure has been described in languages specific to structural features and/or methodological acts, it is to be understood that the present disclosure defined in the appended claims is not necessarily limited to the specific features or acts described above. Rather, the specific features and acts described above are disclosed as example forms of implementing the claims.

I/We claim:

1. A method comprising:
obtaining a number of residues of a protein structure;
generating a protein backbone of the protein structure in a first scale; and
generating, based on the number and the protein backbone, the protein structure in a second scale, the second scale being larger than the first scale.

2. The method of claim 1, wherein generating a protein backbone of the protein structure in a first scale comprises:
generating a first guiding feature in the first scale; and
generating, based on the first guiding feature and a first noise feature, the protein backbone in the first scale.

3. The method of claim 2, wherein generating, based on the number and the protein backbone, the protein structure in the second scale comprises:
determining, by upsampling the protein backbone, a first upsampled feature in the second scale;
generating, based on the first upsampled feature, a second guiding feature in the second scale; and
generating, based on the second guiding feature and a second noise feature, the protein structure.

4. The method of claim 3, wherein generating, based on the second guiding feature and the second noise feature, the protein structure comprises:
determining, based on the second guiding feature and the second noise feature, a denoising vector, wherein the denoising vector indicates a denoising direction and a denoising speed;
iteratively performing the following operations until a preset stopping condition is met:
determining the denoised second noise feature by denoising the second noise feature according to the denoising vector; and
updating, based on the second guiding feature and the denoised second noise feature, the denoising vector; and
taking the denoised second noise feature as the protein structure, wherein the denoised second noise feature comprises three-dimensional coordinates of a plurality of carbon α atoms.

5. The method of claim 4, wherein determining the denoised second noise feature by denoising the second noise feature according to the denoising vector comprises:
determining, based on the denoising vector and the second noise feature, an initial noise feature; and
determining, based on the initial noise feature and random noise, the denoised second noise feature.

6. The method of claim 1, wherein both the first noise feature and the second noise feature belong to Gaussian noise, a dimension of the first noise feature is the same as a dimension of the protein backbone in the first scale, and a dimension of the second noise feature is the same as a dimension of the protein structure.

7. The method of claim 1, wherein generating, based on the number and the protein backbone, the protein structure in the second scale comprises:
determining, based on the protein backbone in the first scale, one or more intermediate protein backbones in one or more intermediate scales; and
generating the protein structure based on the first protein backbone, the one or more intermediate protein backbones, the number and the second noise feature.

8. The method of claim 1, further comprising:
obtaining a third protein backbone in a third scale; and
generating, based on the third protein backbone, a second protein structure.

9. The method of claim 1, further comprising:
obtaining a third protein structure, wherein the third protein structure includes a first portion;
determining a plurality of reference backbones by downsampling the third protein structure;
determining an edited first protein backbone by replacing a partial backbone of the protein backbone in the first scale corresponding to the first portion with a partial backbone of a reference backbone in the first scale corresponding to the first portion; and
generating a fourth protein structure based on the edited first protein backbone.

10. The method of claim 1, wherein the method is performed by a model, and the method further comprises:
obtaining a protein structure sample;
determining a plurality of reference backbones in a plurality of scales by downsampling the protein structure sample; and
training the model based on the plurality of reference backbones and the protein structure sample.

11. The method of claim 10, wherein training the model based on the plurality of reference backbones and the protein structure sample comprises:
determining a plurality of upsampled features for training by upsampling the plurality of reference backbones;
generating one or more predicted protein backbones and a predicted protein structure based on the plurality of upsampled features for training, and a plurality of noise features for training;
determining a loss based on the plurality of reference backbones, the protein structure sample, the one or more predicted protein backbones and the predicted protein structure; and
updating parameters of the model based on the loss.

12. The method of claim 11, wherein determining the plurality of upsampled features for training by upsampling the plurality of reference backbones comprises:
determining a plurality of adjusted reference backbones by adding noise to the plurality of reference backbones; and
determining the plurality of upsampled features for training by upsampling the plurality of adjusted reference backbones.

13. The method of claim 11, wherein the plurality of noise features for training are determined based on the plurality of reference backbones, the protein structure sample and a plurality of Gaussian noise.

14. An electronic device comprising:
at least one processor; and
at least one memory storing instructions that, when executed by the at least one processor, cause the electronic device at least to:
obtain a number of residues of a protein structure;
generate a protein backbone of the protein structure in a first scale; and
generate, based on the number and the protein backbone, the protein structure in a second scale, the second scale being larger than the first scale.

15. The electronic device of claim 14, wherein the instructions to generate a protein backbone of the protein structure in a first scale, further cause the electronic device at least to:
generate a first guiding feature in the first scale; and
generate, based on the first guiding feature and a first noise feature, the protein backbone in the first scale.

16. The electronic device of claim 15, wherein the instructions to generate, based on the number and the protein backbone, the protein structure in the second scale, further cause the electronic device at least to:
determine, by upsampling the protein backbone, a first upsampled feature in the second scale;
generate, based on the first upsampled feature, a second guiding feature in the second scale; and
generate, based on the second guiding feature and a second noise feature, the protein structure.

17. The electronic device of claim 16, wherein the instructions to generate, based on the second guiding feature and the second noise feature, the protein structure, further cause the electronic device at least to:
determine, based on the second guiding feature and the second noise feature, a denoising vector, wherein the denoising vector indicates a denoising direction and a denoising speed;
iteratively perform the following operations until a preset stopping condition is met:
determining the denoised second noise feature by denoising the second noise feature according to the denoising vector; and
updating, based on the second guiding feature and the denoised second noise feature, the denoising vector; and
take the denoised second noise feature as the protein structure, wherein the denoised second noise feature comprises three-dimensional coordinates of a plurality of carbon α atoms.

18. The electronic device of claim 17, wherein the instructions to determine the denoised second noise feature by denoising the second noise feature according to the denoising vector, further cause the electronic device at least to:
determine, based on the denoising vector and the second noise feature, an initial noise feature; and
determine, based on the initial noise feature and random noise, the denoised second noise feature.

19. The electronic device of claim 14, wherein both the first noise feature and the second noise feature belong to Gaussian noise, a dimension of the first noise feature is the same as a dimension of the protein backbone in the first scale, and a dimension of the second noise feature is the same as a dimension of the protein structure.

20. A non-transitory computer-readable storage medium comprising program instructions for causing an apparatus to:
obtain a number of residues of a protein structure;
generate a protein backbone of the protein structure in a first scale; and
generate, based on the number and the protein backbone, the protein structure in a second scale, the second scale being larger than the first scale.
